(19) United States
(12) Plant Patent Application Publication
(10) Pub. No.: US 2018/0077840 P1
(43) Pub. Date: Mar. 15, 2018

(54) Peperomia plant named 'PICCOLO BANDA'

(50) ;

(71) Applicant: Obed Jacob Smit, Rijksweg (NL)

(72) Inventor: Obed Jacob Smit, Rijksweg (NL)

(21) Appl. No.: 15/330,398

(22) Filed: Sep. 15, 2016

(51) Int. Cl. A01H 5/12

(52) U.S. Cl. PLT/373

(57) ABSTRACT

A new cultivar of Peperomia plant named ‘Piccolo Banda’ that is characterized by small heart shaped leaves and light green leaves with brown veins on the upper surface.

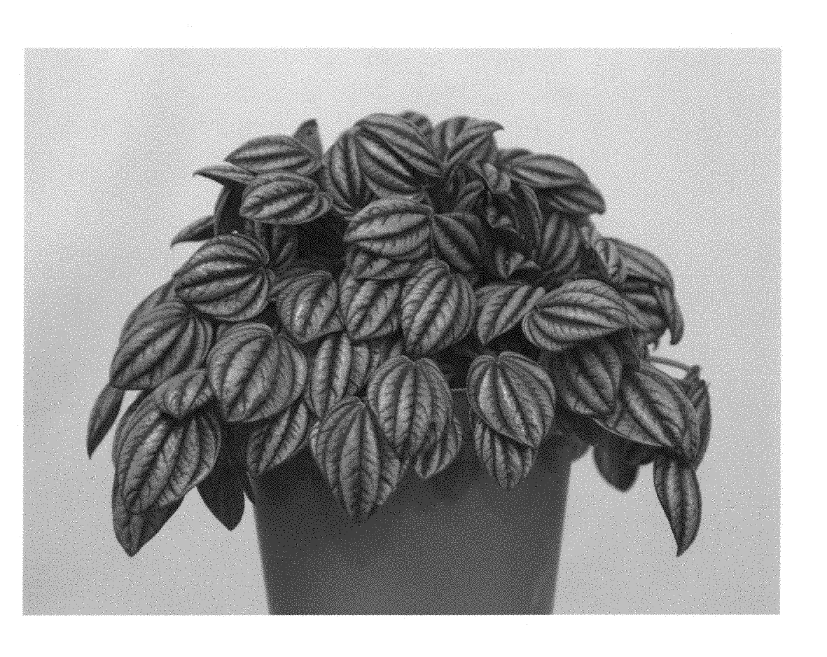

Botanical classification: Peperomia albovittata.

Variety denomination: ‘Piccolo Banda’.

### BACKGROUND OF THE INVENTION

The present invention relates to a new and distinct cultivar of Peperomia plant botanically known as Peperomia albovittata and hereinafter referred to by the cultivar name ‘Piccolo Banda’.

‘Piccolo Banda’ originated from the crossing of the female or seed parent, an unnamed Peperomia albovittata cultivar and the male or pollen parent, an unnamed Peperomia albovittata cultivar. The crossing was conducted in 2013 in Sappemeer, Netherlands. The resulting seeds were subsequently planted and grown. The cultivar ‘Piccolo Banda’ was selected by the inventor in 2014 in a controlled environment as a single plant within the progeny of the stated cross in a cultivated area of Sappemeer, Netherlands.

Asexual reproduction of the new cultivar ‘Piccolo Banda’ first occurred by leaf cuttings in 2014 in Sappemeer, Netherlands. Since that time, under careful observation, the unique characteristics of the new cultivar have been uniform, stable and reproduced true to type in successive generations of asexual reproduction.

### SUMMARY OF THE INVENTION

The following represent the distinguishing characteristics of the new Peperomia cultivar ‘Piccolo Banda’. These traits in combination distinguish ‘Piccolo Banda’ as a new and distinct cultivar apart from other existing varieties of Peperomia known by the inventor.

1. Peperomia ‘Piccolo Banda’ exhibits small heart shaped leaves.
2. Peperomia ‘Piccolo Banda’ exhibits light green leaves with brown veins on the upper surface.

The closest comparison cultivars are Peperomia ‘Eden Rosso’ (U.S. Plant Pat. No. 24,379) and Peperomia ‘Napoli Nights’ (co-pending U.S. Plant patent application). ‘Piccolo Banda’ is distinguishable from ‘Eden Rosso’ by the following characteristics:

1. Peperomia ‘Piccolo Banda’ exhibits light green leaves with brown veins on the upper surface. In comparison, the upper leaf surface of ‘Eden Rosso’ is green.
2. Peperomia ‘Piccolo Banda’ exhibits leaves having a lower surface that is light green. In comparison, the leaf lower surface of ‘Eden Rosso’ is red.

‘Piccolo Banda’ is distinguishable from ‘Napoli Nights’ by the following characteristics:

1. Peperomia ‘Piccolo Banda’ exhibits leaves that are wider than the leaves of ‘Napoli Nights’.
2. Peperomia ‘Piccolo Banda’ exhibits light green leaves with brown veins on the upper surface. In comparison, the leaf upper surface veins of ‘Napoli Nights’ are green in color.

‘Piccolo Banda’ is distinguishable from the female parent plant, by the following characteristics:

1. The leaves of ‘Piccolo Banda’ are smaller than the leaves of the female parent plant.
2. Peperomia ‘Piccolo Banda’ exhibits light green leaves with brown veins on the upper surface. In comparison, the upper surface of the leaves of the female parent plant are green.

‘Piccolo Banda’ is distinguishable from the male parent plant by the following characteristics:

1. The leaves of ‘Piccolo Banda’ are smaller than the leaves of the male parent plant.
2. Peperomia ‘Piccolo Banda’ exhibits light green leaves with brown veins on the upper surface. In comparison, the upper surface of the leaves of the male parent plant are brown-green.

### BRIEF DESCRIPTION OF THE DRAWING

The accompanying photograph illustrates the distinguishing traits of Peperomia ‘Piccolo Banda’. The photograph shows an overall view of a 33 week old plant. The photograph was taken using conventional techniques and although colors may appear different from actual colors due to light reflectance, it is as accurate as possible by conventional photographic techniques.

### BOTANICAL DESCRIPTION OF THE PLANT

The following is a detailed description of the new Peperomia cultivar named ‘Piccolo Banda’. Data was collected in Sappemeer, Netherlands from 33 week old plants grown in a glass greenhouse in 12 cm. diameter containers. The time of year was Winter and the temperature range was 18-25 degrees Centigrade during the day and 15-18 degrees Centigrade at night. The light level was natural light level. No photoperiodic treatments or growth retardants were used. Color determinations are in accordance with The Royal Horticultural Society Colour Chart 2015 edition, except where general color terms of ordinary dictionary significance are used. The growing requirements are similar to the species. ‘Piccolo Banda’ has not been tested under all possible conditions and phenotypic differences may be observed with variations in environmental, climatic, and cultural conditions, however, without any variance in genotype.
Botanical classification: Peperomia albovittata ‘Piccolo Banda’.
Annual or perennial: Perennial.
Parentage: ‘Piccolo Banda’ resulted from the crossing of the female parent, an unnamed Peperomia albovittata cultivar and the male parent an unnamed Peperomia albovittata cultivar.
Plant type: Potted plant.
Growth habit: Rosette.
Plant shape: Flattened globe shaped.
Suitable container size: 7 cm. pots or larger.
Plant height: 10.8 cm.
Plant width: 24.5 cm.
Vigor: Moderate.
Low temperature tolerance: 5° Centigrade.
High temperature tolerance: 40° Centigrade.
Propagation: Leaf cuttings.
Time to initiate roots (summer): 10 days at 18 to 20° C.
Time to initiate roots (winter): 14 days at 18 to 20° C.
Time to produce a rooted cutting (summer): 80 days at 18 to 20° C.
Time to produce a rooted cutting (winter): 100 days at 18 to 20° C.
Growth rate: Moderate.
Crop time: Approximately 30 to 35 weeks in Sappemeer, Netherlands.
Root system: Fibrous.
Plant fragrance: None.
Stem:
   Branching habit.—Short thick stems branching into rosettes of leaves.
   Number of main stems per plant.—Average 18.
   Number of lateral branches per plant.—Average 72.
   Main stem dimensions.—1.7 cm. in length and 1.4 cm. in width.
   Internode length.—0.1 cm.
   Stem shape.—Rounded.
   Stem texture.—Smooth.
   Stem pubescence.—Absent.
   Stem angle.—0 degrees.
   Stem strength.—Strong.
   Stem color (young).—145B.
   Stem color (mature).—197A.
   Internode color.—197A.
Foliage:
   Leaf arrangement.—Rosette.
   Compound or single.—Single.
   Quantity of leaves per main stem.—Average 26.
   Leaf shape.—Ovate.
   Leaf aspect.—Concave.
   Leaf apex.—Acute.
   Leaf base.—Cordate.
   Leaf dimensions.—4.4 cm. in length and 3.7 cm. in width.
   Texture.—Glabrous (both surfaces).
   Leaf luster.—Upper surface glossy, lower surface matte.
   Pubescence.—Absent.
   Leaf margin.—Entire.
   Venation pattern.—Parallel, furrowed.
   Young leaf color (upper surface).—147B, veins between N186C and 200A.
   Young leaf color (lower surface).—146D.
   Mature leaf color (upper surface).—189A.
   Mature leaf color (lower surface).—Between 138C and 147D.
   Vein color (upper surface).—Between N186C and 200A.
   Vein color (lower surface).—147B.
Petiole:
   Petiole dimensions.—8.4 cm. in length and 0.3 cm. in diameter.
   Petiole texture.—Glabrous.
   Petiole luster.—Slightly glossy.
   Petiole pubescence.—Absent.
   Petiole strength.—Low.
   Petiole color.—176A.
Flower: ‘Piccolo Banda’ has not produced flowers to date.
Fruit and seed: ‘Piccolo Banda’ has not produced fruit or seed to date.
Disease and pest resistance: Disease and pest resistance has not been observed.

1. A new and distinct variety of Peperomia plant named ‘Piccolo Banda’ as described and illustrated.
